(19) United States
(12) Patent Application Publication
(10) Pub. No.: US 2011/0125194 A1
(43) Pub. Date: May 26, 2011

(54) ARTICULATED INSERT

(75) Inventors: Gregor Anwand, Duderstadt (DE); Alexander Hardt, Duderstadt (DE); Olaf Kroll-Orywahl, Gottingen (DE); Maximilian Segl, Duderstadt (DE); Norbert Schimek, Kirchworbis (DE)

(73) Assignee: OTTO BOCK HEALTHCARE GMBH, Duderstadt (DE)

(21) Appl. No.: 13/002,744

(22) PCT Filed: Jul. 13, 2009
(86) PCT No.: PCT/DE09/00970

§ 371 Date: Jan. 5, 2011

(30) Foreign Application Priority Data
Jul. 24, 2008 (DE) 10 2008 034 750.7

(51) Int. Cl. A61B 17/84

(52) U.S. Cl. 606/300

(57) ABSTRACT

The invention relates to an articulated insert (1), comprising an upper connecting means (2) for fastening to a first orthopedic component and a lower connecting means (2) for fastening to a second orthopedic component that can be pivoted relative to the first orthopedic component, and further comprising a tensile force-transmitting joining element (4) between the receiving devices (2, 3), wherein the joining element (4) has a planar, flexible, dimensionally stable material section (6, 6′), which is helically twisted in the longitudinal extension thereof.

The invention relates to an articulated insert for an orthopedic device, with an upper connecting means for fastening to a first orthopedic component and with a lower connecting means for fastening to a second orthopedic component that is mounted pivotably relative to the first orthopedic component, and with a tensile force-transmitting joining element between the two connecting means. The articulated insert is provided in particular for orthoses, e.g. an ankle orthosis.

EP 712 468 B1 describes a composite flexure unit with two fastening devices for fastening to two orthosis components that are pivotable relative to each other. Arranged between the two fastening devices there is a load-bearing element that transmits tensile force and that is made from a fiber with a low coefficient of friction, said fiber being wound around end sleeves. The load-bearing element is freely bendable in every direction. The fastening devices and the load-bearing element are encapsulated in an elastic plastic. Alternatively, the load-bearing element is designed as a strip of fabric.

Moreover, cardanic joint devices or chain connections can be provided between two orthopedic elements in order to join these to each other. The cardanic bearing can be designed, for example, as a ball joint or a traditional cardan bearing with intersecting axes. The chain connections can be embodied by ball chains, which are placed in corresponding split bearings. Such solutions can lead to displacement of the ball joints and, as a whole, provide unsatisfactory guiding of the components relative to each another.

The object of the present invention is to make available an articulated insert with which it is possible to permit, between two orthopedic elements, in particular orthosis elements, a flexible connection with a resilient action, wherein a pivoting movement is intended to be possible about one or more defined axes. According to the invention, this object is achieved by an articulated insert having the features of Claim 1. Advantageous embodiments and developments of the invention are set forth in the dependent claims.

The articulated insert according to the invention, with an upper connecting means for fastening to a first orthopedic component and with a lower connecting means for fastening to a second orthopedic component that is mounted pivotably relative to the first orthopedic component, and with a tensile force-transmitting joining element between the two connecting means, is such that the joining element has a flexurally elastic, dimensionally stable material section, which has a cross section with different axial moments of inertia, and the axis of the smaller axial moment of inertia is parallel at least in part to the pivot axis of the orthopedic components. By means of the flexural elasticity of the material section, it is possible to make available a spring action, such that improved guiding and coordination of the two orthopedic components relative to each other is possible. In the case of an orthosis insert, the limb that is to be supported is better guided in this way. In addition, a flexible connection of the two orthopedic components is achieved without the distances between the connection points substantially changing, as a result of which a movement of the two orthopedic components relative to each other is substantially avoided. The arrangement of the axes of the area moments of inertia has the effect that a slight and desired flexion or shifting of the components about an axis is permitted, whereas a bending about the second axis is, as desired, made difficult by means of the greater area moment of inertia and, consequently, bending resistance.

In a development of the invention, the material section is twisted in its longitudinal extent. By means of the helical configuration of the planar material section, it is possible that the respective bending axis, which lies within the planar material section, that is to say runs parallel to the width of the material section, can be set precisely, and in particular the position of the bending axis can be precisely determined by means of the twisting being effected in such a way that the line with the lowest area moment of inertia and with the lowest modulus of elasticity is located where the pivot axis between the two orthopedic components is intended to lie.

In a development of the invention, the material section is twisted through at least 90° in order to permit a bending movement about two axes oriented perpendicular to each other. The material section, which is shaped as a strip, is preferably twisted through integral multiples of 90°, in particular 180° or 360°, so as to be able to provide two or three bending axes oriented perpendicular to each other.

The material section is preferably made of sheet metal or plastic, the choice of material being made so as to provide sufficient elasticity and spring-back. The permanent flexural strength of the material section or of the entire joining element must also be such as to ensure that the articulated insert can at all times function reliably.

In a development of the invention, the joining element has two separate material sections, with form-fit elements which are arranged thereon and which engage in each other in a manner transmitting tensile force and permit a pivotability about an axis. By means of the pivotability about an axis, it is possible to provide a more or less free mobility of the two connecting means and therefore of the orthopedic devices, e.g. orthosis components, without having to take into consideration the restoring force on account of the spring action of the material sections. This can be favorable for particular purposes if a mobility is to be possible freely about one axis, for example in order to define a preferred direction of movement, while other directions of movement have to be effected counter to a bending resistance.

The material section is preferably configured in one layer, but it can also be composed of a laminate or a composite component. Alternatively to a single-layer configuration of the material section, provision is made that said material section is composed of a closed loop and has a corresponding shape such that the connection between the two connecting means is ensured while at the same time providing sufficient flexural elasticity.

It can likewise be possible that the entire joining element is designed as a closed loop with mutually opposite material sections, such that the joining element as a whole can be made from a tube section or hose section, e.g. in the configuration of the joining element as a fiber-reinforced plastic component.

The connecting means preferably have a sleeve receiving screws or similar fastening means, so as to permit simple fastening of the connecting means to the respective orthopedic devices. The sleeve can be formed in one piece on the joining element and in particular in one piece with the material section as a deformation section, as a winding or as a loop-end section, such that no additional joining measures have to be taken. Alternatively, the sleeve and also the connecting means can be fastened separately to the joining element, for example in order to be able to achieve high pressure stability of the fastening by a separate choice of material.

Since the joining element, particularly when configured as a sheet metal or glass-fiber reinforced component, can have sharp edges, provision is made for it to be encased, in particular by a flexible elastomer which, in addition to a function of protecting the joining element and the connecting means from external influences, also provides contact protection for the person wearing the orthopedic device and additionally improves handling. In addition, an additional amount of restoring force is afforded by the elastomer, in addition to the flexural elasticity by the joining element or the material section.

Illustrative embodiments of the invention are explained in more detail below with reference to the attached figures, in which:

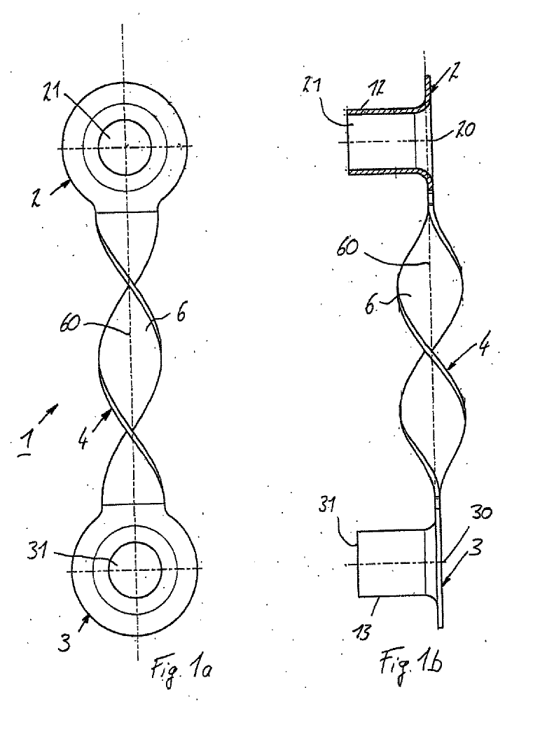

FIGS. 1a and 1b show a plan view and a side view of a first variant;

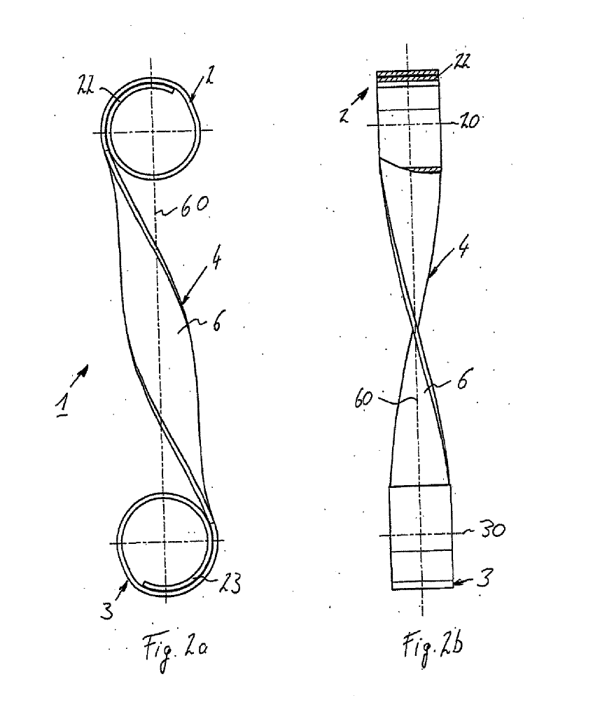

FIGS. 2a and 2b show a second variant with integrally formed connecting means;

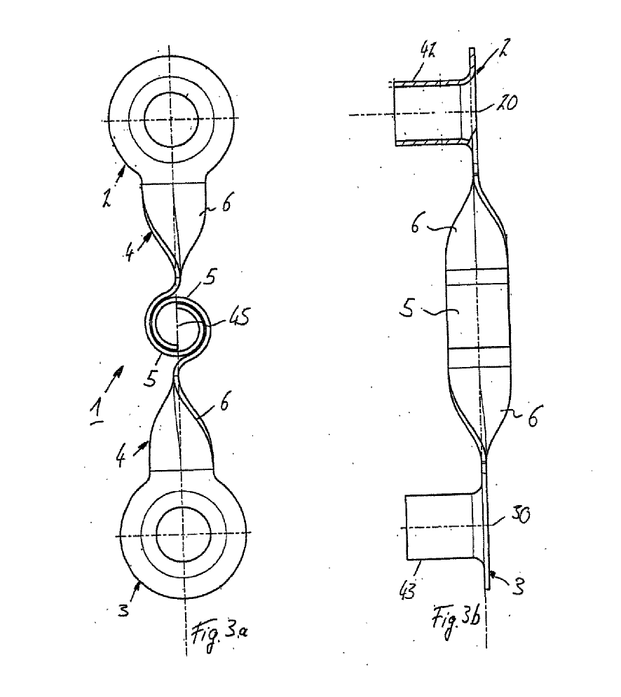

FIGS. 3a and 3b show a two-part variant of the articulated insert with two joining elements; and

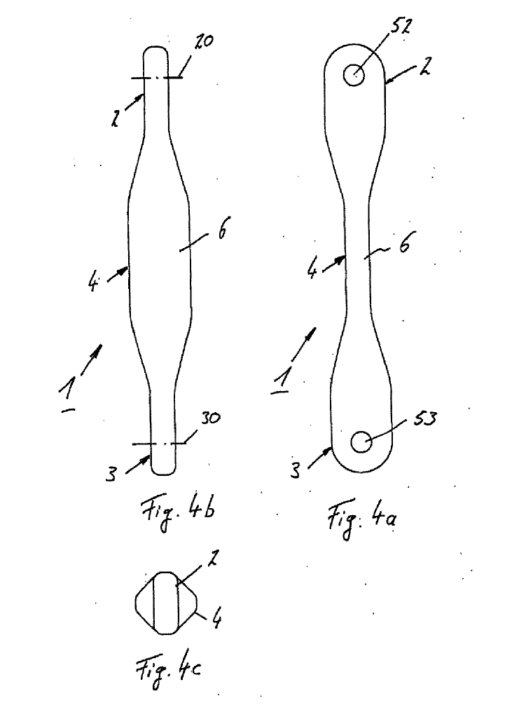

FIGS. 4a to 4c show a further variant of the articulated insert.

FIG. 1a shows a plan view of an articulated insert 1 with an upper connecting means 2 and a lower connecting means 3 for fastening to two orthopedic components (not shown), for example orthosis components. The lower connecting means 3 can, for example, be fastened to a foot part of an ankle orthosis, while the upper connecting means 2 is fastened to the lower-leg element of the ankle orthosis. The fastening is preferably done using screws, which are guided through openings 21, 31 in the connecting means 2, 3. Sleeves 12, 13 are formed on the connecting means 2, 3, which sleeves 12, 13 can be seen in the side view in FIG. 1b. The sleeves 12, 13 are formed in one piece with the connecting means 2, 3 and extend substantially perpendicular with respect to planar flanges that can serve as bearing areas for screws. The screws are inserted along the center axes 20, 30 of the sleeves 12, 13, such that the articulated insert 1 can be fastened securely yet releasably on the orthopedic components.

Between the connecting means 2, 3 there is a joining element 4, which is composed of a helically twisted material strip 6. The material strip 6 is twisted through 360° along its longitudinal extent 60 and forms a helix. The material strip 6, preferably a sheet metal strip, is dimensionally stable and ensures a defined distance between the two connecting means 2, 3. The material strip 6 can take up tensile forces and compressive forces and is flexurally elastic, such that the two orthopedic components (not shown) remain oriented relative to each other in a stable manner in a non-applied state. If the two connecting points are moved relative to each other, for example twisted about the center axes 20, 30, a bending takes place at two points within the material strip 6, specifically where the narrow sides of the material strip intersect the center axes 60. A bending thus takes place on the upper quarter and lower quarter of the material strip 6.

Referring to FIG. 1b, a bending takes place perpendicular to the longitudinal extent 6 at the center between the two connecting means 2, 3, again where the narrow side of the material strip 6 intersects the center axis 60, because it is there that the lowest area moment of inertia and the lowest modulus of elasticity are present. In this way, it is possible to make available a resilient articulated insert 1 which, in the embodiment according to FIGS. 1a and 1b, is produced in one piece from a sheet metal component. The sleeves 12, 13 can be formed in the context of a compression forming method, similar to a deep-drawing method. The two connecting means 2, 3 are then twisted through 360° relative to each other, such that a helical configuration of the joining element 1 is achieved. The resilience is achieved through the nature of the material, and the positioning of the respective preferred pivot axes is obtained through the arrangement and configuration of the helical course of the material section 6, with pivot axes preferably being provided which run parallel to the center axes 20, 30 of the sleeves 12, 13, as is shown in FIG. 1a, and perpendicular thereto, as can be seen in FIG. 1b.

FIGS. 2a and 2b show a variant of the invention in which an articulated insert 1 is likewise shown, which is composed of a single material strip 6, in the present case a sheet metal strip. The connecting means 2, 3 are formed by winding up the end areas, such that corresponding sleeves 22, 23 for receiving screws or other fastening elements are formed which have a sufficient dimensional stability. The material strip 6 is likewise helical, in the present case twisted through 180°, such that the material strip 6 bears tangentially on the sleeves 22, 23 on different sides of the center line 60. A preferred bending takes place about a bending axis perpendicular to the plane of the drawing, as is shown in FIG. 2b, at the point where the narrow side of the material strip 6 intersects the center line 60. A pivotability about a pivot axis parallel to the center axes 20, 30 of the sleeves 22, 23 is possible only when much greater force is applied, since the material strip 6 is not correspondingly oriented.

A further variant is shown in FIGS. 3a, 3b, in which the articulated insert 1 is designed in two parts. Here, two identically shaped sheet metal components are provided which each have a connecting means 2, 3 and a joining element 4 in the form of an integrally attached material strip 6. At the end of the material strips 6, there are form-fit elements 5 in the shape of hook-like, round bend sections, which are designed such that the width of the material strip 6 are oriented parallel to the sleeves 42, 43. This is achieved by the material strip 6 being twisted through 90°. The form-fit elements 5 are inserted into each other and, by virtue of the configuration of the form-fit elements 5 as partial circles, pivot about a pivot axis 45 arranged centrally between the connecting means 2, 3. The pivot axis 45 extends perpendicularly from the plane of the drawing and runs parallel to the center axes 20, 30 of the connecting means 2, 3. A flexural elasticity is therefore made available about this pivot axis 45 not by the spring resistance of the material sections 6 but if appropriate by other elastic means that surround the articulated insert 1, for example encapsulating silicone or another elastomer. A flexural elasticity in the area of the material sections 6 is effected close to the respective transition to the connecting means 2, 3 or to the flange areas of the connecting means 2, 3.

FIGS. 4a to 4c show a further variant of an articulated insert 1. The articulated insert 1 has upper and lower connecting means 2, 3 with recesses 52, 53 arranged around center axes 20, 30. FIG. 4a shows an articulated insert 1 in a side view, and FIG. 4b shows an articulated insert 1 in a side view rotated through 90°. The joining element 4 is formed in one piece between the two connecting means 2, 3 and has a flat material section 6 which is in the form of a strip with a substantially rectangular cross section and which is arranged with a constant orientation between the connecting means 2, 3. The rectangular cross-sectional shape of the material section 6 results in different degrees of axial area moments of inertia and, consequently, different degrees of bending resistance. In FIG. 4, the orientation of the material section 6 is chosen such that the axis of the lower area moment of inertia is oriented parallel to the center axes 20, of the connecting means 2, 3, with the result that, when fastening is effected in the usual way by screws or the like parallel to the axis of movement of the two orthopedic components that are to be joined, the axis of the small area moment of inertia runs parallel to the pivot axis of the orthosis components.

FIG. 4c shows the articulated insert 1 in a plan view. It will be seen here that the connecting means 2 also have a substantially rectangular cross section, wherein the two longitudinal axes of the rectangular cross sections of the connecting means 2, 3 and of the material section 6 are oriented perpendicular to each other. The transition area between the receiving devices 2, 3 and the material section 6 represents a continuous transition between the cross sections, although non-continuous transitions can also be provided between the connecting means 2, 3 and the material section 6. The articulated insert 1 can be produced in one piece from a suitably elastic material, for example a metal or plastic or a composite material, and the articulated insert 1 can likewise be encased with a layer of plastic.

All the variants in FIGS. 1 to 4 can be encapsulated with a silicone or another elastomer. Instead of a sheet metal configuration, it is likewise possible for the joining element 1 to be produced from an elastic plastic or a composite material, for example a fiber-reinforced, strip-shaped material.

1-13. (canceled)

14. An articulated insert (1) with an upper connecting means (2) for fastening to a first orthopedic component and with a lower connecting means (3) for fastening to a second orthopedic component that is mounted pivotably relative to the first orthopedic component, and with a tensile force-transmitting joining element (4) between the receiving devices (2, 3), wherein the joining element (4) has a flexurally elastic, dimensionally stable material section (6, 6′), which has a cross section with different axial area moments of inertia, and the axis of the smaller axial area moment of inertia is parallel at least in part to the pivot axis of the orthopedic components, characterized in that the material section (6, 6′) is twisted in its longitudinal extent.

15. The articulated insert as claimed in claim 14, characterized in that the material section (6, 6′) is twisted through at least 90°.

16. The articulated insert as claimed in claim 14 or 15, characterized in that the material section (6, 6′) is twisted through integral multiples of 90°, in particular through 180° or 360°.

17. The articulated insert as claimed in one of the preceding claims, characterized in that the material section (6, 6′) is made of sheet metal or plastic.

18. The articulated insert as claimed in one of the preceding claims, characterized in that the joining element (4) has two separate material sections (6), with form-fit elements (5) which are arranged thereon and which engage in each other in a manner transmitting tensile force and permit a pivotability about an axis (45).

19. The articulated insert as claimed in one of the preceding claims, characterized in that the material section (6, 6′) is configured in one layer.

20. The articulated insert as claimed in one of claims 14 through 18, characterized in that the material section (6, 6′) is formed from a closed loop.

21. The articulated insert as claimed in one of claims 14 through 17, characterized in that the joining element (4) is designed as a closed loop with mutually opposite material sections (6, 6′).

22. The articulated insert as claimed in one of the preceding claims, characterized in that the connecting means (2, 3) have a sleeve (12, 13, 22, 23, 32, 33, 42, 43).

23. The articulated insert as claimed in one of the preceding claims, characterized in that the connecting means (2, 3) are fastened separately to the joining element.

24. The articulated insert as claimed in one of claims 14 through 22, characterized in that the connecting means (2, 3) is formed in one piece with the material section as a deformation section, winding or loop-end section.

25. The articulated insert as claimed in one of the preceding claims, characterized in that the joining element (4) is encapsulated, in particular by a flexible elastomer.
